(19) United States
(12) Patent Application Publication
(10) Pub. No.: US 2026/0224518 A1
(43) Pub. Date: Aug. 6, 2026

(54) ORGANIC TRACE MINERALS OF L-LYSINE OR A DERIVATIVE THEREOF AND THEIR USE IN HUMAN OR ANIMAL FEED

(71) Applicant: METABOLIC EXPLORER, SAINT-BEAUZIRE (FR)

(72) Inventors: Tristan CHALVON-DEMERSAY, Paris (FR); Josselin LE COUR GRANDMAISON, Clichy (FR); Antoine REVOLTE, Amiens (FR)

(73) Assignee: METABOLIC EXPLORER, SAINT-BEAUZIRE (FR)

(21) Appl. No.: 19/147,544

(22) PCT Filed: Jan. 12, 2024
(86) PCT No.: PCT/EP2024/050735

§ 371 Date: Jul. 11, 2025

(30) Foreign Application Priority Data
Jan. 13, 2023 (EP) 23305053.3

(51) Int. Cl. A61K 31/198; A23K 20/142; A23K 20/20; A23K 50/30; A23K 50/75; A61K 33/04; A61K 33/06; A61K 33/24; A61K 33/26; A61K 33/30; A61K 33/32; A61K 33/34; A61P 3/02; C07F 1/08; C07F 3/02; C07F 3/06; C07F 13/00; C07F 15/02; C07F 15/06

(52) U.S. Cl. CPC A61K 31/198; A23K 20/142; A23K 20/30; A23K 50/30; A23K 50/75; A61K 33/04; A61K 33/06; A61K 33/24; A61K 33/26; A61K 33/30; A61K 33/32; A61K 33/34; A61P 3/02; C07F 1/08; C07F 3/02; C07F 3/06; C07F 13/005; C07F 15/025; C07F 15/065

(57) ABSTRACT

The invention relates to OTM of amino acid and minerals, the unique structure of rendering them useful in human and animal feed so as to improve in vivo bioavailability and assimilation of mineral sources.

### FIELD OF THE INVENTION

The present invention concerns the domain of human and animal feed, in particular diet of livestock animals such as poultry and pigs. More specifically, the present invention concerns organic trace minerals of amino acid and minerals, the unique structure of rendering them useful in human and animal feed so as to improve in vivo bioavailability and assimilation of mineral sources.

### BACKGROUND OF THE INVENTION

Animal nutrition is a domain in constant evolution, both for pet animals or farm animals. In particular, farm or livestock animals constitute valuable source of quality nutrition and economic development across the world.

Thus, the diet of animals has to be adapted in order to favorize growth, and more generally to sustain their good health and performances, so as to provide the diversity of goods and services such as meat, milk, eggs, hides, fibers, feathers etc., according to the cases.

Adequate provision of amino acids in animal diets has been shown to enhance the performances of farm animals. Furthermore, a dietary supplementation with essential amino acids allows the use of low-protein diets, and therefore has been used for many years. Traditionally, amino acids are classified as nutritionally “essential” or “non-essential”. Nutritionally essential amino acids are so termed because the carbon skeleton of these amino acids cannot be synthesized in the biological system, and thus must be supplied through the diet. Each animal species has its own specificities; for example, pigs require only ten essential amino acids, although poultry require twelve essential amino acids to be supplied in its diet.

In addition, mineral sources such as iron, copper, zinc or manganese, are involved in structural, physiological, catalytic, and regulatory functions in animals. Thus, their inclusion as supplements in animal diets is necessary for a multitude of reasons. Mineral supplementation is becoming a major issue in livestock due to the rising concern on sustainability and minerals output in the environment. Providing these compounds within an organic or complex structure under the form of organic trace minerals (OTM) increases the bioavailability of minerals compared to their inorganic forms, notably oxides and sulfates. OTM are nutritional elements added to animal diets in micro quantities, usually mixed with the food in order to balance or adjust feed intakes.

Several different types of OTM are currently available, based on the type of ligand (amino acid, peptide, polysaccharide or organic acid) used to bond with the mineral. These types of products have been shown to have different mineral binding properties.

Better solutions are always needed to still increase the bioavailability of mineral sources while limiting their spreading in the environment in the context of human and animal nutrition.

### SUMMARY OF THE INVENTION

The present invention relates to OTM based on amino acids or a derivative thereof, in particular L-amino acids, with minerals, useful as bioavailable mineral sources in human and animal feed.

The OTM organic trace minerals according to the present invention are OTM of L-lysine or a derivative thereof with minerals, wherein the L-lysine or the derivative thereof and the mineral are in a molar ratio of less than 1:1, preferably in a molar ratio L-lysine or derivative thereof:mineral between 1:2 and 1:3. More preferably, a single type of minerals is complexed with L-lysine, thus meaning that each complex comprises only one type of minerals.

In particular, the OTM according to the present invention exhibits an morphous structure. Also, there is a covalent bond between the mineral and the amino acid, preferably L-lysine or a derivative thereof, in the OTM of the present invention.

More specifically, the OTM of the invention comprise a mineral selected in the group consisting of zinc, copper, iron, manganese, calcium, magnesium, cobalt, selenium, preferably zinc, copper, iron and manganese.

One other aspect of the present invention relates to a feed supplement comprising one or more OTM as described above.

An advantageous aspect of the present invention is specifically the use of the OTM or the feed supplement comprising thereof in human or animal feed. Such a use may be in livestock animal feed, in pet nutrition/feed or in aquaculture feed. Preferably, the use is in livestock animal feed, and in particular poultry feed.

The present invention also relates to the use of the OTM as described above for stimulating growth performance, preferably in the starter phase, and reducing mineral excretion. To this end, the OTM with L-lysine or a derivative thereof according to the present invention are particularly advantageous, since they may be supplemented at a dose providing only half of the amount of minerals compared to the amount of minerals contained in inorganic forms without negatively impacting performance of animals.

Additionally, the present invention is related to the use of L-lysine or a derivative thereof in an OTM with minerals, comprised in a food composition for improving in vivo bioavailability and assimilation of mineral sources, wherein the L-lysine or the derivative thereof and the mineral are in a molar ratio of less than 1:1, and preferably in a molar ratio between 1:2 and 1:3, in the OTM.

### BRIEF DESCRIPTION OF THE FIGURES

FIG. 1 is an illustrative process description for preparing complexes according to the present invention.

FIG. 2. is RMN ¹H (400 MHz) spectra in D₂O of a complex L-lysine with zinc according to the invention (A) and of the comparative Gly-Zn (B).

FIG. 3. is RMN ¹H (400 MHz) spectra in D₂O of a complex L-lysine with iron according to the invention (A) and of the comparative Gly-Fe (B).

FIG. 4. is RMN ¹H (400 MHz) spectra in D₂O of a complex L-lysine with copper according to the invention (A) and of the comparative Gly-Cu (B).

FIG. 5. is RMN ¹H (400 MHz) spectra in D₂O of a complex L-lysine with manganese according to the invention (A) and of the comparative Gly-Mn (B).

FIG. 6. is RMN 13C (125 MHz) spectra in D₂O of a complex L-lysine with zinc according to the invention (A) and of the comparative Gly-Zn (B) and of L-lysine (C).

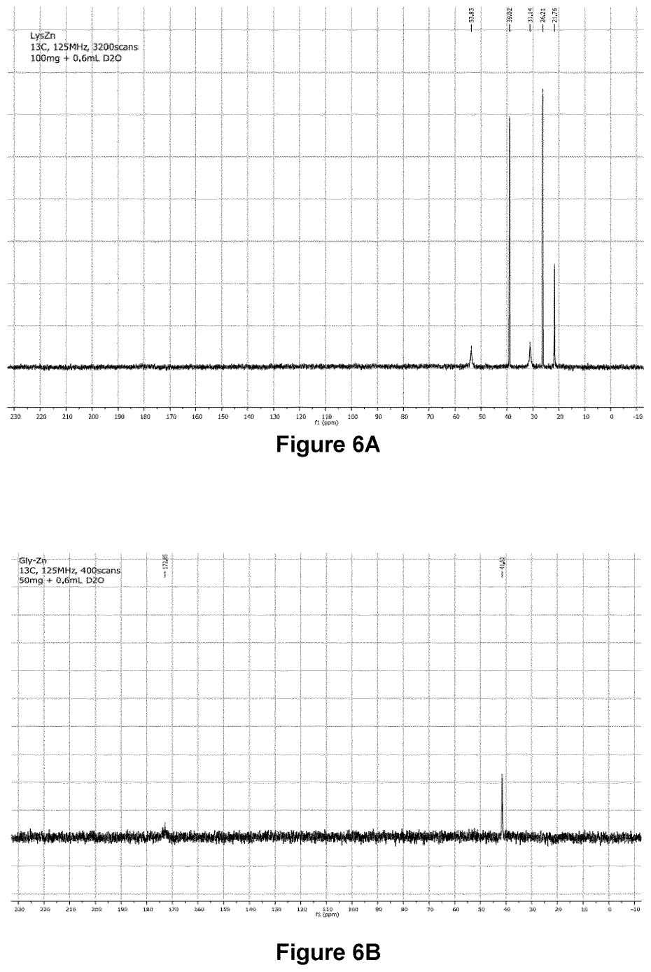

FIG. 7. is RX diffractogram of a complex L-lysine with zinc according to the invention (A) and of the comparative Gly-Zn (B).

FIG. 8. is RX diffractogram of a complex L-lysine with iron according to the invention (A) and of the comparative Gly-Fe (B).

FIG. 9. is RX diffractogram of a complex L-lysine with copper according to the invention (A) and of the comparative Gly-Cu (B).

FIG. 10. is RX diffractogram of a complex L-lysine with manganese according to the invention (A) and of the comparative Gly-Mn (B).

FIG. 11. represents the body weight of broiler fed with the OTM of the invention compared to the comparative complexes based on glycine, measured at d12 (A) and d35 (B).

FIG. 12. represents the Zn content (A) and Fe content (B) measured in the faeces of broilers fed with the OTM of the invention compared to the comparative complexes based on glycine (full dose and half dose).

FIG. 13. represents the Cu content (A) and Mn content (B) measured in the faeces of broilers fed with the OTM of the invention compared to the comparative complexes based on glycine (full dose and half dose).

### DETAILED DESCRIPTION OF EMBODIMENTS OF THE INVENTION

Other characteristics and advantages of the invention will now be described in detail in the following description which is given with reference to the appended figures.

In a first aspect, the present disclosure relates to OTM based on amino acids or a derivative thereof, in particular L-amino acids, with minerals, useful as bioavailable mineral sources in human and animal feed.

As well known by the person skilled in the art, amino acids designate organic compounds that contain both an amino (—NH2) and carboxylic acid (—COOH) functional group, and are the basic building blocks of proteins. Today 20 amino acids have been listed: Alanine, Arginine, Asparagine, Aspartic Acid, Cysteine, Glutamic acid, Glutamine, Glycine, Histidine, Isoleucine, Leucine, Lysine, Methionine, Phenylalanine, Proline, Serine, Threonine, Tryptophan, Tyrosine and Valine.

Among them, nine amino acids are essential for humans; ten are essential for pigs (in young age) and for poultry: Arginine, Methionine, Histidine, Phenylalanine, Isoleucine, Threonine, Leucine, Tryptophan, Lysine and Valine.

The present disclosure is related to OTM based on amino acids chosen among the 20 amino acids as listed above, and in particular based on the essential amino acids for the considered animal species. A more specific disclosure is related to OTM based on amino acid(s) chosen among the group consisting of tyrosine, proline, histidine, valine, cysteine, isoleucine, leucine, tryptophan, arginine, lysine, or any of their derivatives. More preferably, it is disclosed OTM based on any proteinogenic amino acid, preferably selected in the group consisting of tryptophan, valine, leucine, isoleucine, arginine, cysteine or cystine, histidine, lysine, or any of their derivatives. Most preferred amino acids are L type.

In the present invention, OTM is based on a lysine, especially L-lysine.

### Organometallic Complexes According to the Invention

The OTM according to the present invention are OTM of L-lysine or a derivative thereof with minerals, wherein the L-lysine or the derivative thereof and the mineral are in a molar ratio of less than 1:1, preferably in a molar ratio L-lysine or derivative thereof:mineral between 1:2 and 1:3.

Preferably, a single type of minerals is complexed with L-lysine, thus meaning that each complex comprises only one type of minerals.

Conversely, no amino acid other than L-lysine or a derivative thereof is present in the OTM of the present invention. Also, no amino acid other than L-lysine or a derivative thereof is associated with the type of minerals in the OTM of the present invention. Furthermore, the OTM of the present invention are more preferably based on L-lysine and not on L-lysine derivative or any other amino acid or derivative than L-lysine, and where the L-lysine and the mineral are in a molar ratio of less than 1:1, preferably in a molar ratio L-lysine:mineral between 1:2 and 1:3.

The molar ratio is determined by any method known by the skilled person in the art.

The OTM of the present invention are highly promising due to their unique structure and the utilization of an essential amino acid: L-Lysine to bind to the minerals. The inventors have characterized the link between L-Lysine and minerals in these OTM, the structure of which is crucial to protect the mineral from undesirable chelate formation as well as improving mineral bioavailability.

By “a derivative (there) of”, results from reaction of amino group or carboxy group of an amino acid or from the replacement of any hydrogen of amino acid by a heteroatom. It is intended to mean particularly a hydroxy analog, seleno analog, oxidated analog of an amino acid, and the like. Specifically, where related to L-lysine, “derivative (there) of” refers to hydroxylysine, phenyllysine, acetonyllysine, carbamoylmethyllysine, methyllysine, isodesmosine, carboxyethyllysine, pyrrolysine, carboxymethyllysine, cadaverine, 5-aminovalerate, carnitine, α-aminoadipate.

In particular, the OTM according to the present invention exhibits an amorphous structure and not a crystalline form.

Also, there is a covalent bond between the mineral and the amino acid, preferably L-lysine or a derivative thereof, in the OTM of the present invention.

Preferably, the mineral is divalent. More specifically, the OTM of the invention comprise a mineral selected in the group consisting of zinc, copper, iron, manganese, calcium, magnesium, cobalt, selenium, preferably zinc, copper, iron and manganese.

### Preparation of the OTM of the Invention

The OTM according to the present invention may be obtained by any methods for complexing amino acids and mineral sources, which are well known by the skilled person in the art.

The OTM of the invention are preferably prepared using spray-drying and granulation methods well known in the art. For example, appropriate device to be used for manufacturing the complex according to the invention is described in EP2161075. More preferably, the complexes of the invention are prepared by a fluidized bed granulation process.

In particular, the amino acid is in a solution preferably an aqueous solution. Preferably, mineral sources are mineral salts, for example among oxides, sulfates and the like.

Particularly advantageous OTM according to the invention are those obtained based on L-lysine and mineral sulfates, in particular L-lysine and zinc sulfate, L-lysine and copper sulfate, L-lysine and iron sulfate or L-lysine and manganese sulfate.

Advantageously, the main particulars to prepare the complex of the invention are mixing an aqueous solution of L-lysine, preferably in water only, with mineral sulfate, preferably with zinc sulfate, copper sulfate, iron sulfate, manganese sulfate, before applying a spray drying granulation method by fluidized bed.

One other aspect of the present invention relates to a feed supplement comprising one or more OTM as described above.

The feed supplement may be under any formulation type, such as liquid, powder, premix and the like. The most appropriate formulation is selected in view of the host susceptible to receive the supplement, in particular the most adapted to the diet form with which the supplement is to be used (granulates, pellets, mash feed, kibbles, liquids, etc.).

### Administration of the OTM of the Invention

An advantageous aspect of the present invention is specifically the use of the OTM or the feed supplement comprising thereof in human or animal nutrition.

Such a use may be in livestock animal feed, in pet nutrition/feed or in aquaculture feed. Preferably, the use is in livestock animal feed, and in particular poultry feed.

“Livestock animals” are the domesticated animals raised in an agricultural setting to provide labor and produce diversified products for consumption such as meat, eggs, milk, fur, leather, and wool. This term as used herein thus refer a broad definition so as to refer to any population of animals kept by humans for a useful, commercial purpose, such as horse, donkey, bovine, sheep, goat, pig, rabbit, poultry, and the like.

Poultry designates any type of bird that humans raise for food, feathers or work. Poultry includes notably chickens, turkeys, geese and ducks.

In a specific embodiment of the invention, the OTM or the feed supplement comprising thereof is used in feeding animals chosen among pigs and poultry, preferably poultry, and in particular chickens/broilers.

The present invention is also related to the use of the complex described above in pet animal feed. Pet animals may be various and at least includes dogs and cats.

Aquaculture refers to the breeding of algae or animals in an aquatic environment. It is related to agriculture as concerning the breeding of fish, crustaceans and mollusks and the culture of algae. In aquaculture, the use of the OTM or the feed supplement comprising thereof is preferably used in food for animals in an aquatic environment, and more preferably fish, crustaceans and mollusks.

In the implementation of the invention, the OTM of the invention can be administered by any route to animals or humans, in particular via oral route.

In a preferred embodiment of the invention, the OTM are administered orally, and in particular are incorporated into the diet of animals or humans.

For administration to non-human animals, the OTM or the feed composition comprising thereof may be added to the animal's feed or drinking water. Optionally, one skilled in the art will recognize that animal feed and drinking products may be formulated such that the animal takes in an effective amount of OTM of the present invention via their diet in view of their need in terms of minerals and amino acid.

The complexes of the present invention may also be formulated as food or drink supplements for humans. More specifically in humans and in pet animals, the complexes of the invention are used for providing the most appropriate nutritional needs regarding amino acids, in particular L-lysine, and regarding minerals, in particular zinc, copper, iron, manganese, calcium, magnesium, cobalt, selenium, and preferably zinc, copper, iron and manganese.

An efficient amount of said OTM or the feed composition comprising thereof is incorporated into the diet of animals, i.e., an amount allowing to obtain the desired effects as detailed below.

In particular, OTM are incorporated into the diet in an amount corresponding to the dose of minerals usually supplemented in inorganic forms in the diet of a given animal.

The present invention also relates to the use of the OTM as described above for stimulating growth performance, preferably in the starter phase, and reducing mineral excretion. To this end, and as demonstrated in the present application, the OTM with L-lysine or a derivative thereof according to the present invention are particularly advantageous, since they may be supplemented at a dose providing only half of the amount of minerals compared to the amount of minerals contained in inorganic forms without negatively impacting performance of animals. The phrase “stimulating growth performance” refers to an improvement of the body weight, and/or increased feed conversion, ideally combined with an increased ratio of meat or the valuable part of the animal versus the body structure, such as skeleton, which cannot be valuable, compared to a control group.

In this context, by “starter phase of growth”, it is intended to mean the 7 to 14 first days following hatching.

The relevant skilled persons of the art, in particular farmers and veterinarians or physicians, know well the biological effects to be achieved and how to assess them, in order to identify any significant deficiency or improvements to be obtained. All these biological effects are representative of the “performance” of the farm animals, which is an indication of how well a certain animal performed in his/her genetic economic valuable traits over its lifetime.

Additionally, the present invention is related to the use of L-lysine or a derivative thereof in an OTM with a mineral, comprised in a food composition for improving in vivo bioavailability and assimilation of mineral sources, wherein the L-lysine or the derivative thereof and the mineral are in a molar ratio of less than 1:1, and preferably in a molar ratio between 1:2 and 1:3, in the OTM.

All the preferred embodiments mentioned above apply to this additional aspect of the invention.

### EXAMPLES

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### Example 1. Preparation of OTM According to the Present Invention

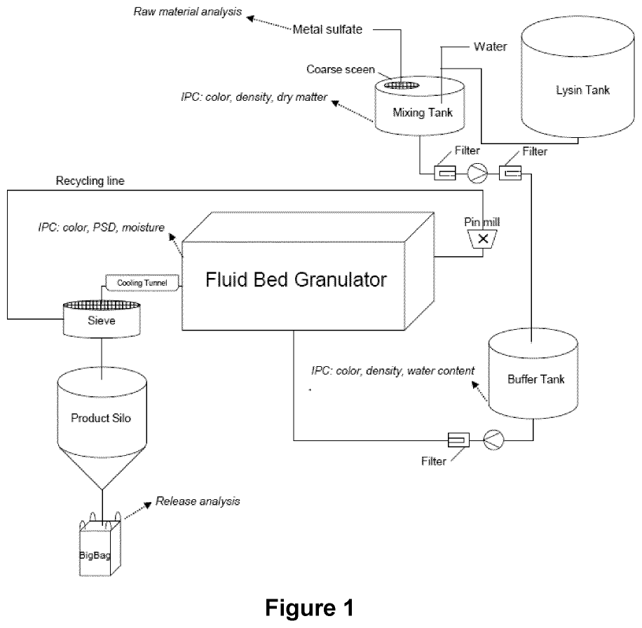

The process description is summarized in FIG. 1.

A solution of L-lysine (50% aq.) was charged to the mixing tank, where the mineral sulfate in the powder form was added (Cu sulfate, Fe sulfate, Zn sulfate or Mn sulfate). The quantities used as summarized in Table 1 below, the minerals being the following: Copper sulfate pentahydrate, Zinc sulfate monohydrate, Manganese sulfate monohydrate and Iron sulfate heptahydrate.

TABLE 1

| | | | |
|---|---|---|---|
| Lysinate | Lysinate | Lysinate | Lysinate |
| Fe | Mn | Cu | Zn |

Raw materials
(in g/100 g)

| | | | | |
|---|---|---|---|---|
| L-Lysine LLB50 | 28 | 28 | 27 | 27 |
| Mineral | 66 | 40 | 57 | 40 |
| Water | 6 | 32 | 16 | 32 |

Raw materials
(in g/100 g as
dry matter basis)

| | | | | |
|---|---|---|---|---|
| L-Lysine | 28 | 28 | 27 | 27 |
| Mineral | 72 | 72 | 73 | 73 |

Raw materials
(in mole/100 g as
dry matter basis)

| | | | | |
|---|---|---|---|---|
| L-Lysine | 0.19 | 0.19 | 0.19 | 0.19 |
| Mineral | 0.47 | 0.47 | 0.46 | 0.45 |
| Molar ratio | 2.42 | 2.44 | 2.43 | 2.40 |

The mixing tank, buffer tank and heat tracing were set to 70-80° C. for Zn-, Fe- and Cu-formulations (no heating for Mn-formulation). The agitation in the mixing tank was approximately 30 minutes, then all was transferred to the buffer tank, before a continuous atomization of spray solution and granulation in fluid bed granulator. The inlet air temperature was up to 230° C. and the product temperature of about 95° C.

During all the process, the following were performed:
controls of spray solution (color, precipitation, . . . ) and product (water content, color, particle size distribution);
continuous discharge, cooling in cooling channel and fractionation on sieve,
recycling of fines and coarse particles into process.

The obtained product was discharged into bigbag and sampling for release analysis (mineral content, particle size distribution, bulk density, water content) was carried out.

### Example 2. Characterization of the OTM of the Invention

### 2.1. Molar Ratio

TABLE 2
analyses by ICP for minerals [symbol] chromatography for Lysine

| | | | |
|---|---|---|---|
| Lysinate | Lysinate | Lysinate | Lysinate |
| Fe | Mn | Cu | Zn |

Content
(% of final product)

| | | | | |
|---|---|---|---|---|
| L-Lysine | 25 | 25 | 24 | 24 |
| Mineral | 23 | 20 | 29 | 25 |

Content
(mole/100 g of
final product)

| | | | | |
|---|---|---|---|---|
| L-Lysine | 0.17 | 0.17 | 0.17 | 0.16 |
| Mineral | 0.42 | 0.36 | 0.45 | 0.39 |
| Ratio | 2.5 | 2.1 | 2.7 | 2.4 |

In order to characterize the OTM of the invention based on L-Lysine and the respective mineral Zn, Fe, Cu and Mn, the different tests described below were performed and the results compared to the results obtained with the four following complexes based on glycine with the same minerals, commercialized by other companies on the market:
Gly-Zn: Zincate(1-), diaqua(glycinato-kO)[sulfato(2-)-kO]—, hydrogen, (T-4)
Gly-Fe: Ferrate(2-), hexaaqua[μ-(glycinato-kO:kO′)](glycinato-kO)bis[sulfato(2-)-kO]di-, dihydrogen, stereoisomer
Gly-Cu: Cuprate(1-), diaqua(glycinato-kO)[sulfato(2-)-kO]—, hydrogen
Gly-Mn: Manganate(1-), (glycinato-N,O)[sulfato (2-)-O]—, hydrogen.

### 2.1. Molar Ratio

Analyses by Inductif Coupled Plasma Optical Emission Spectrometry (ICP-OES) to quantify minerals and chromatography to quantify Lysine are realized under experimental conditions well known by the man of the art. The results in Table 3 below show a molar ratio between lysine and the mineral between 1:2 and 1:3.

TABLE 3

| | | | |
|---|---|---|---|
| Lysinate | Lysinate | Lysinate | Lysinate |
| Fe | Mn | Cu | Zn |

Content
(% of final product)

| | | | | |
|---|---|---|---|---|
| L-Lysine | 25 | 25 | 24 | 24 |
| Mineral | 23 | 20 | 29 | 25 |

Content
(mole/100 g of
final product)

| | | | | |
|---|---|---|---|---|
| L-Lysine | 0.17 | 0.17 | 0.17 | 0.16 |
| Mineral | 0.42 | 0.36 | 0.45 | 0.39 |
| Ratio | 2.5 | 2.1 | 2.7 | 2.4 |

### 2.2. Covalent Bound

In order to characterize the link between L-Lysine and the respective mineral in the complexes of the invention, the following tests were performed: NMR¹H; NMR¹³C and XRD on powder.

### Material & Methods

The NMR 1H and 13C spectra were obtained with a Bruker Avance III HD 400 MHz spectrometer equipped with an autosampler. The analyses were performed in D₂O (Eurisotop, stored in the refrigerator). The spectra of protons 1H protons are calibrated using the peak of the protonated solvent residue in the deuterated solvent. Impurities possible: Acetone (2.22 ppm).

The UV-Vis analysis of the compounds was performed with an Agilent Cary 5000 UV-vis-NIR spectrophotometer in single beam measurement from 200 to 800 nm. The samples were dissolved in distilled water then transferred in a quartz cell of volume 1.5 mL and optical path 1 cm.

### Results

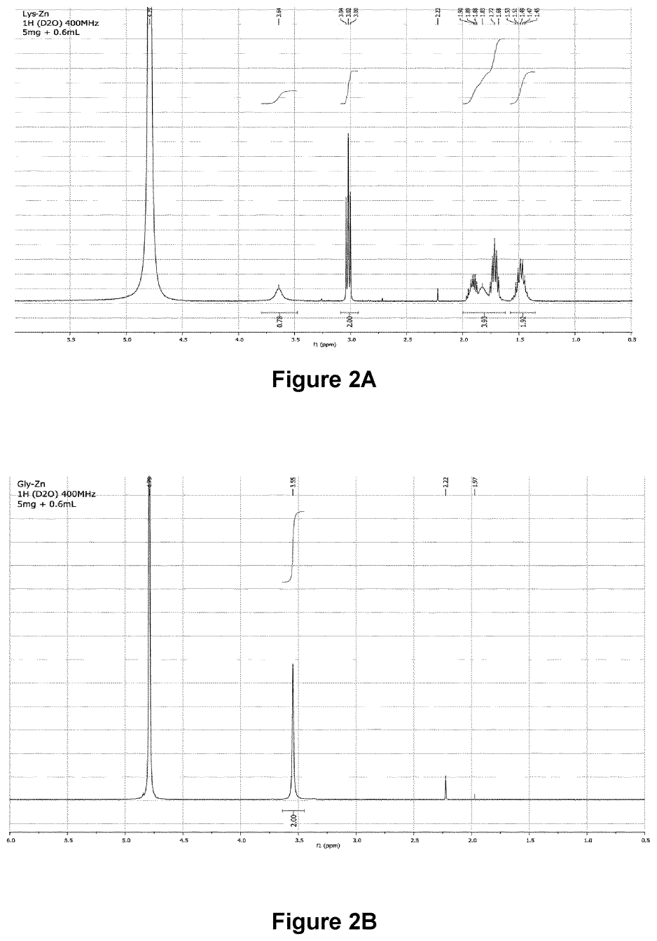

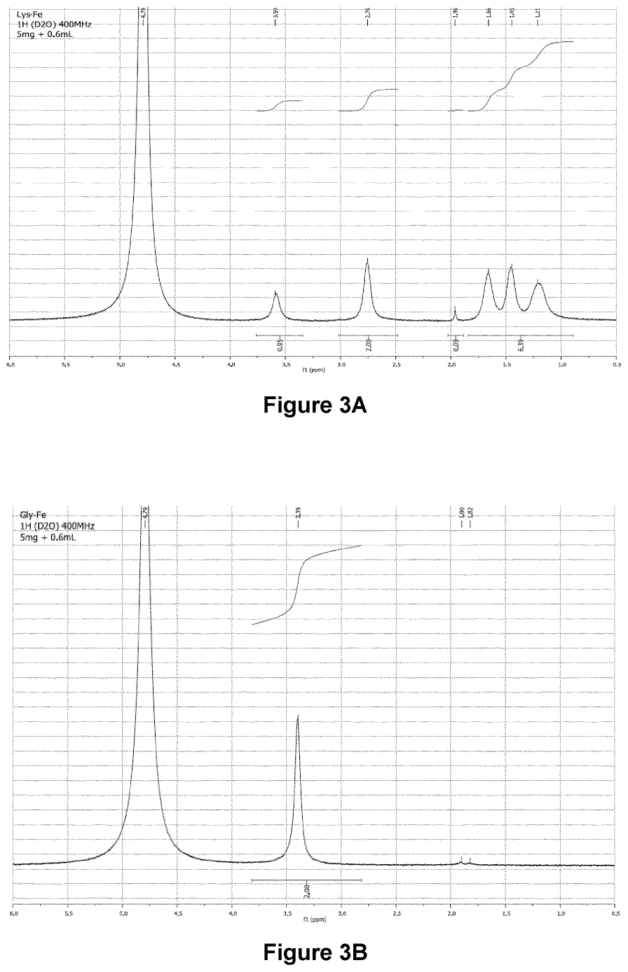

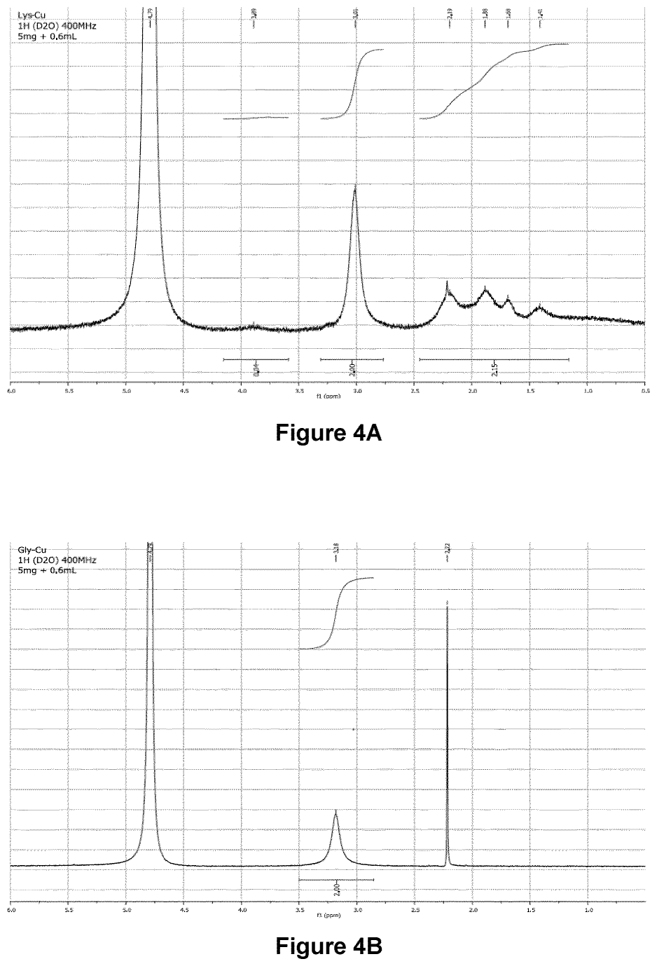

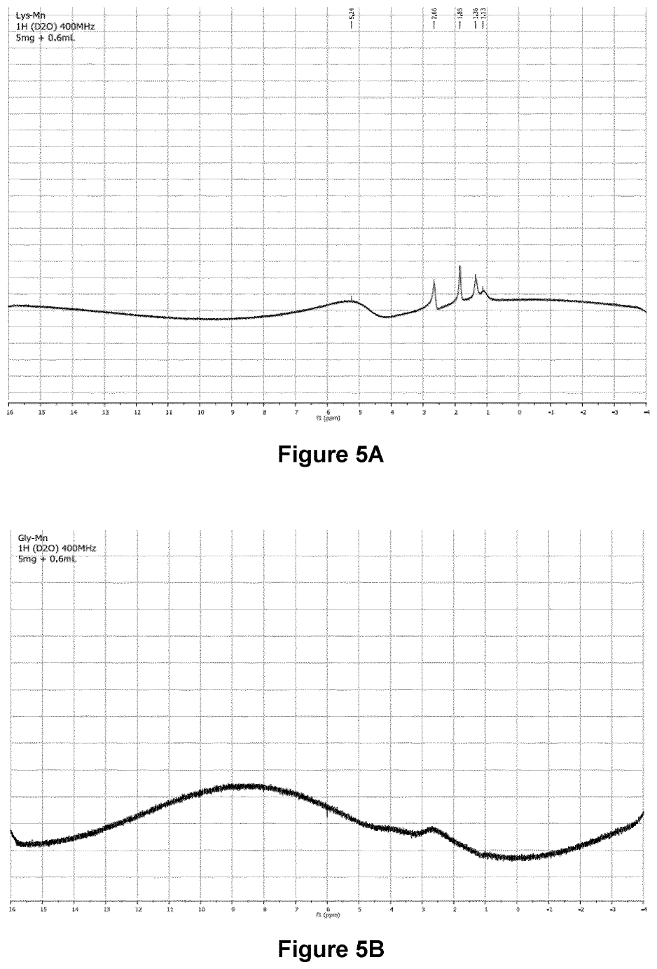

The NMR¹H spectra of the OTM of the invention (L-Lysine_Zn, L-Lysine_Fe, L-Lysine_Cu and L-Lysine_Mn), compared to those of the comparative Gly-Zn Gly-Fe, Gly-Cu, Gly-Mn are in FIGS. 2 to 5, respectively.

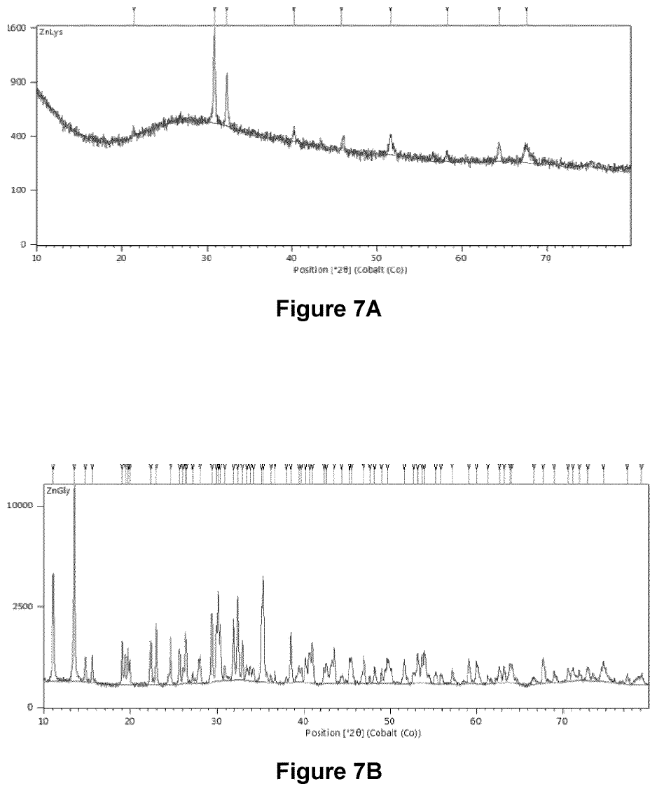

As an example, the ¹³C spectra of L-Lysine_Zn, GLy_Zn and L-lysine are shown in FIG. 7.

NMR 13C indicated a molecular adaptation between the carboxyl group and the amine group of lysine. The molecular adaptation and its multiplicity is characteristic of a coordination between the mineral and the L-Lysine.

NMR 1H, 13C and UV-vis spectro results (data not shown) demonstrated a covalent bond between L-Lysine and minerals for each OTM tested.

### 2.3. Amorphous Structure

### Material & Methods

A Bruker D8 ENDEAVOR autosampler diffractometer was used to analyze the OTM and the comparatives as mentioned above. The diffractograms were then processed using the HighScore software.

### Results

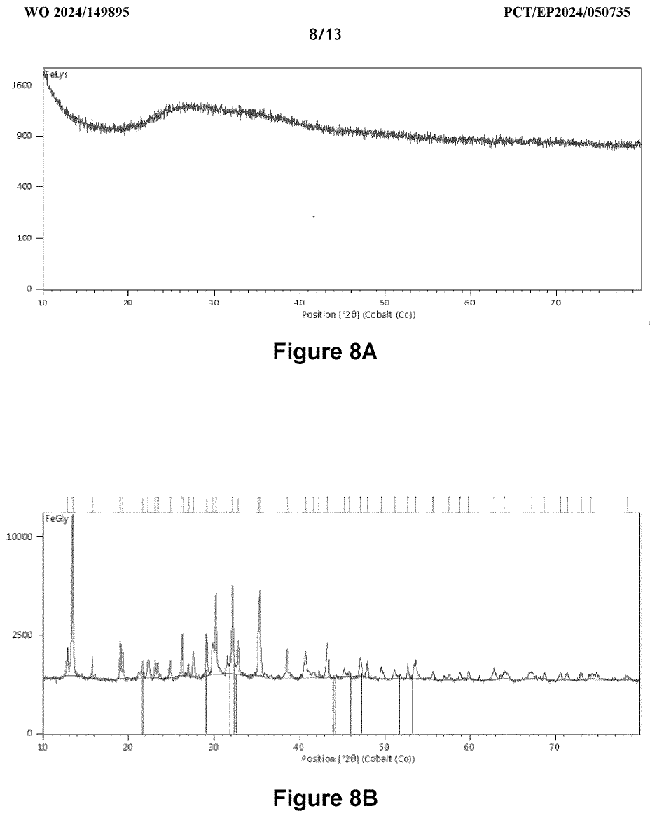

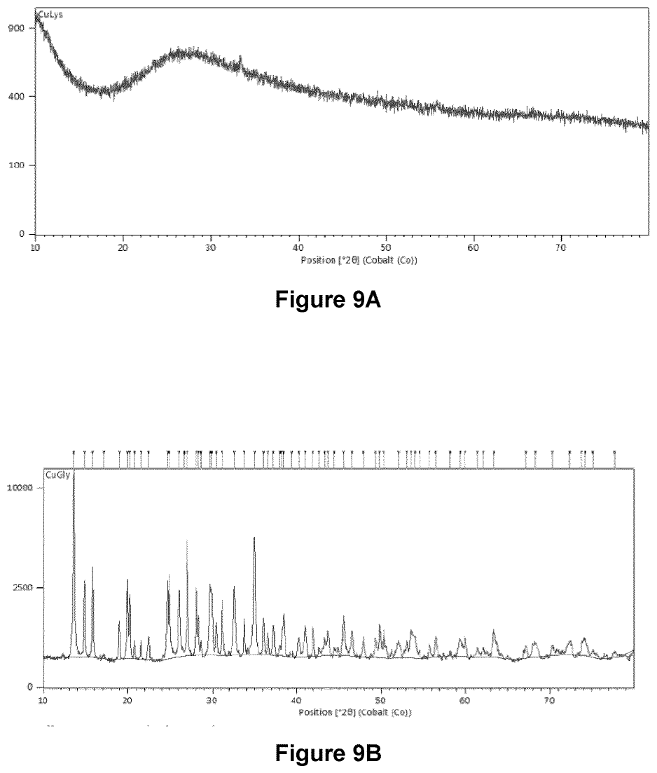

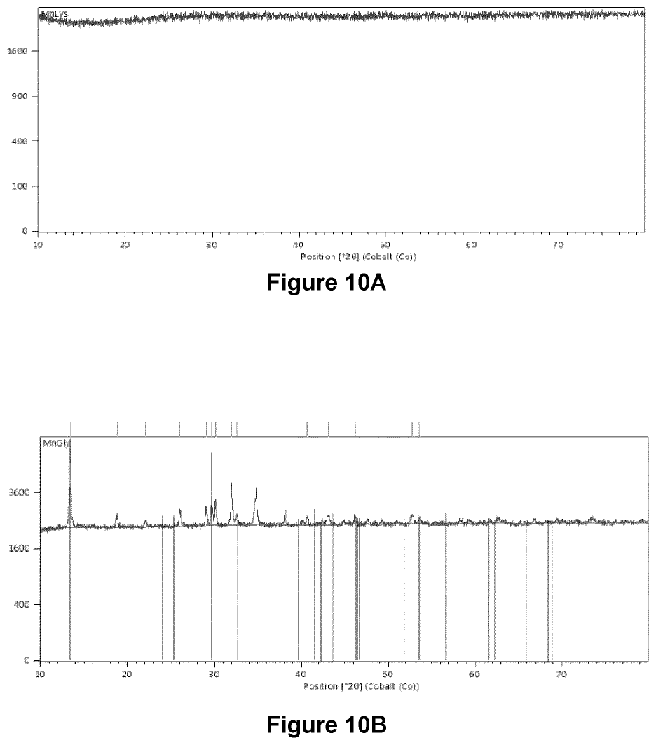

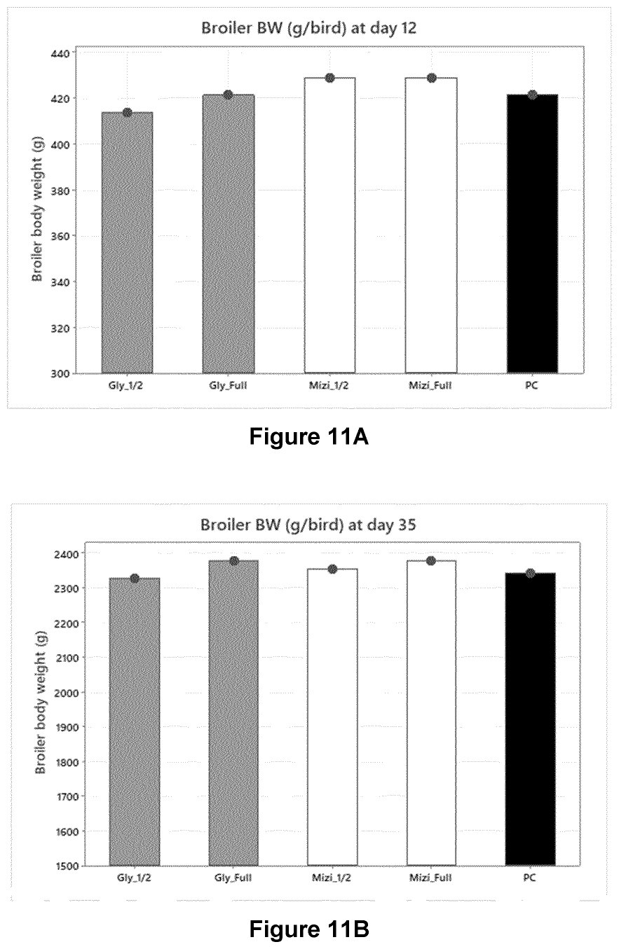

Diffractograms RX of the OTM of the invention (L-Lysine_Zn, L-Lysine_Fe, L-Lysine_Cu and L-Lysine_Mn), compared to those of the comparative Gly-Zn Gly-Fe, Gly-Cu, Gly-Mn are shown in FIGS. 8 to 11.

DRX results clearly indicated the absence of distinct and dissociated mineral and lysine fragments in the complexes. Therefore, the OTM of the invention are not under crystalline form, but rather under amorphous structure.

Altogether, these results show that the OTM according to the invention are unique complexes of L-lysine and minerals, with an amorphous structure and wherein the mineral and amino acid are linked with covalent bonds.

### Example 3. Effects of OTM Administration to Broiler Chickens with Respect to Growth Performance

The response on growth performance of broiler fed with the four OTM of the invention L-Lysine_Zn, L-Lysine_Fe, L-Lysine_Cu and L-Lysine_Mn compared to other source of minerals (sulphates and oxides and the comparatives based on glycine Gly-Zn Gly-Fe, Gly-Cu, Gly-Mn) was evaluated as follows:

### Material & Methods

The experiment took place in the west part of France. Male broilers (n=1000) were randomly allocated into 5×10 pens corresponding to 5 treatments with 10 replicates. Animals were fed experimental diets similar in nutritional constraints and raw material composition, the only difference being the mineral types and dosage as illustrated in the table 4 below.

TABLE 4

| | | Cu | Fe | Zn | Mn |
|---|---|---|---|---|---|
| Treatments | Minerals | (mg/kg) | (mg/kg) | (mg/kg) | (mg/kg) |
| Positive | Sulphates [symbol] | 10 | 30 | 80 | 100 |
| Control | oxides | | | | |
| Mizi_½ | Lysinate | 5 | 15 | 40 | 50 |
| | half dose | | | | |
| Mizi_Full | Lysinate | 10 | 30 | 80 | 100 |
| Gly_½ | Glycinate | 5 | 15 | 40 | 50 |
| | half dose | | | | |
| Gly_Full | Glycinate | 10 | 30 | 80 | 100 |

Animal growth performances were followed until 35 days and at slaughter carcass traits were evaluated.

### Results

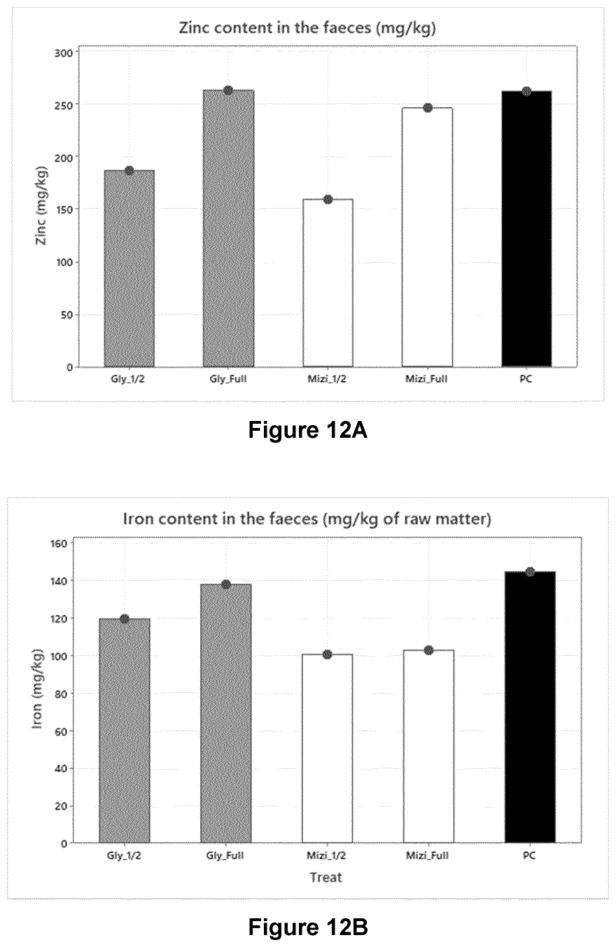

Results are shown in FIG. 12, at d12 (A) and at d35 (B).

At day 12 a tendency for better performance were detected for broiler fed with the combined complexes according to the invention (named Mizi herein) versus other sources. At the end of the fattening period (d35) the difference was only numerical, but a tendency of higher carcass weight and breast to carcass ratio was noticed using the complex Mizi of the invention at full dose (cf. Table 5 below).

TABLE 5

| | Body | Body | Feed | | |
|---|---|---|---|---|---|
| | weight | weight | conversion | Carcass | Breast/ |
| | at d 12 | at d 35 | ratio from | weight | carcass |
| Treatments | (in g/bird) | (in g/bird) | d 0 to d 35 | (g) | ratio (%) |
| Positive | 422 | 2342 | 1.44 | 1558 | 31.2 |
| Control | | | | | |
| Mizi_½ | 429 | 2354 | 1.44 | 1546 | 31.5 |
| Mizi_Full | 429 | 2377 | 1.43 | 1569 | 32.0 |
| Gly_½ | 414 | 2329 | 1.43 | 1512 | 31.6 |
| Gly_Full | 421 | 2378 | 1.43 | 1538 | 31.5 |

These performance results indicated that it is possible to use half dose of minerals without compromising animal performance. Still the best performance was observed using OTM: Complex of the invention (Mizi) or Glycinate at full dose.

### Example 4. Effects of OTM Administration to Broiler Chickens with Respect to Mineral Excretion

### Material & Methods

To assess minerals bioavailability, faeces minerals content was evaluated for copper, iron, zinc and manganese by dosage by optical ICP after mineralization with aqua regia (three parts of concentrated nitric acid and one part of concentrated hydrochloric acid) on digiprep block (https://www.scpscience.com/en/products/categories?id=36)

### Results

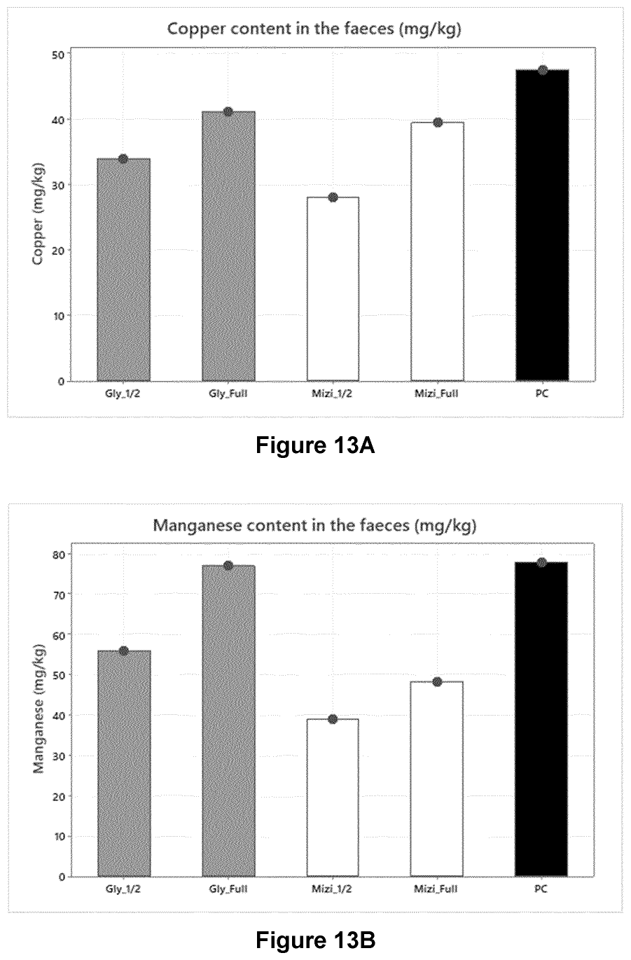

The results obtained in broiler faeces are illustrated in FIG. 13.

Significant lower content of minerals was measured in broiler fed Mizi_Iron and Mizi_Mn half or full dose in comparison to other treatments (p<0.05). For Mizi_Zn and Mizi_Cu, mineral content in the faeces were the lowest when using half dose vs. full dose and the positive control (p<0.05).

Altogether, these results show that the OTM of the invention are highly bioavailable minerals sources that enable broiler to growth better while excreting less.

1. Organometallic complex of L-lysine or a derivative thereof with a mineral, wherein the L-lysine or the derivative thereof and the mineral are in a molar ratio of less than 1:1.

2. Organometallic complex of L-lysine or a derivative thereof with a mineral of claim 1, wherein the L-lysine or the derivative thereof and the mineral molar ratio between 1:2 and 1:3.

3. Organometallic complex of L-lysine or a derivative thereof with a mineral of claim 1, wherein the complex exhibits an amorphous structure.

4. Organometallic complex of L-lysine or a derivative thereof with a mineral of claim 1, wherein the L-lysine or the derivative thereof and the mineral are covalently linked.

5. Organometallic complex of L-lysine or a derivative thereof with a mineral of claim 1, wherein the mineral is selected in the group consisting of zinc, copper, iron, manganese, calcium, magnesium, cobalt and selenium, preferably zinc, copper, iron and manganese.

6. Feed supplement comprising one or more organometallic complex of claim 1.

7. Human or non-human animal feed comprising the organometallic complex of claim 1 or the feed supplement of claim 8 in human or non-human animal feed.

8. The human or non-human feed of claim 7, wherein the feed is a livestock animal feed, pet feed, or feed for aquaculture.

9. The human or non-human feed of claim 8, wherein the feed is a livestock animal feed.

10. A method of for stimulating growth performance and reducing mineral excretion, which comprises feeding the human or non-human animal feed of claim 7 to a human or non-human animal.

11. The method of claim 10, wherein the organometallic complex is supplemented at a dose providing only half of the amount of minerals compared to the amount of minerals contained in inorganic forms without negatively impacting performance of animals.

12. Method for improving in vivo bioavailability and assimilation of mineral sources in a food composition, which comprises feeding an human or non-human animal with a food composition comprising L-lysine or a derivative thereof in an organometallic complex with minerals wherein the L-lysine and the mineral atom are in a molar ratio of less than 1:1.

13. Human or non-human animal feed comprising the feed supplement of claim 6.

14. Human or non-human feed of claim 9, wherein the livestock animal feed is poultry feed.

15. Method of stimulating growth performance of claim 10 wherein the human or non-human animal feed is provided to the human or non-human animal in the starter phase.
